(19) United States
(12) Patent Application Publication
(10) Pub. No.: US 2026/0000301 A1
(43) Pub. Date: Jan. 1, 2026

(54) METHOD AND APPARATUS FOR HIGH-FIDELITY LENSLESS MULTIMODE FIBER-BASED PHOTOACOUSTIC ENDOMICROSCOPY

(71) Applicant: The Hong Kong Polytechnic University, Hung Hom (HK)

(72) Inventors: Puxiang LAI, Hung Hom (HK); Tianting ZHONG, Hung Hom (HK); Shengfu CHENG, Hung Hom (HK); Chi Man WOO, Hung Hom (HK)

(73) Assignee: The Hong Kong Polytechnic University, Hung Hom (HK)

(21) Appl. No.: 18/759,180

(22) Filed: Jun. 28, 2024

(51) Int. Cl. A61B 5/00

(52) U.S. Cl. CPC A61B 5/0095; A61B 5/742; A61B 2560/0223

(57) ABSTRACT

A method and implementing apparatus for high-fidelity lensless multimode fiber-based photoacoustic endomicroscopy. The method includes an improved nonconvex optimization method to achieve non-interferometric calibration of multimode optical fiber that is used for laser scanning imaging. Before imaging, the complex-valued transmission matrix is quickly reconstructed directly from the multimode fiber output intensity measurement without the need for a reference beam. During imaging, wavefront shaping technology is used to perform high-speed laser scanning at the end of the multimode fiber, which excites photoacoustic signals on tissues. The photoacoustic signals are detected in a non-focused manner by a high-sensitivity fiber-optic-ultrasonic-sensor outside the sample. The implementing apparatus includes a wavefront shaping module, a fiber imaging probe, and a photoacoustic signal acquisition and processing module.

### BACKGROUND OF THE INVENTION

### 1. Field of the Invention

An aspect of the invention relates specifically to a method and implementing apparatus for high-fidelity lensless multimode fiber-based photoacoustic endomicroscopy, or “MMF-PAEM”.

### 2. Description of the Related Art

Various imaging methods exist to nondestructively examine living tissue for purposes of diagnostic and research activities. Traditional radiological methods (x-rays, magnetic imaging) require expensive, bulky equipment and can produce hazardous radiation which must be shielded against. In contrast, optical imaging is advantageous due to its non-ionizing radiation, high sensitivity, and low cost, which plays a more and more important role in biomedicine. However, living tissues have variable opaqueness to light and can cause scattering or excessive absorption. To overcome the strong light scattering and absorption by tissues and achieve deep-tissue optical imaging, photoacoustic endoscopic imaging technology has been developed to achieve high-resolution observation of blood vessels and other tissues at depth. Photoacoustic imaging uses a pulsed laser to illuminate biological tissue. The tissue absorbs the light to generate heat: the heat causes the tissue to expand outward to generate sound waves. By detecting these sound waves, which are typically ultrasonic, the light absorption coefficient of the tissue can be determined. Photoacoustic imaging has rich optical absorption contrast and large ultrasound penetration depth. By using endogenous light absorbers such as oxygenated and deoxygenated hemoglobin and lipids or exogenous contrast agents such as nanoparticles, it can generate predetermined signal ranges for blood vessels or other specific tissues. The target tissue thus provides structural, functional and molecular information and has important applications in neuroscience, tissue diagnosis and other fields.

Existing high-resolution photoacoustic endoscopic probes usually integrate laser scanning and photoacoustic signal receiving modules into an imaging probe. The laser scanning either uses a single-mode optical fiber to transmit focused light and the probe's built-in scanning module to perform distal scanning or uses a scanning galvanometer to control the beam for scanning at the proximal side of an optical fiber bundle and coupling into different fiber cores, combined with a micro-objective attached in the end to improve the scanning resolution. The photoacoustic signal detection module usually uses a built-in miniature ultrasonic transducer in the imaging probe itself. This usually requires that the size of the entire probe range from millimeters to centimeters. Although this kind of imaging probe may meet the needs of some medical imaging applications such as gastrointestinal tract and intrauterine cavity imaging, for imaging scenarios such as studying neuro-vascular activities in the brain of small animals, the use of such probes may cause tissue damage which can harm the subject and/or reduce the usefulness of the imaging data. Therefore, a high-resolution photoacoustic endoscope with ultrafine resolution and minimal footprint would be a useful invention.

The core diameter of multimode optical fiber is usually about one hundred microns and can transmit thousands of modes. In theory, it can transmit complex image information for endoscopic imaging. However, multimode optical fiber is a complex medium with inherent modal dispersion and coupling that limit its direct imaging applications. Optical wavefront shaping technology uses a spatial light modulator (SLM) to control the incident wavefront of multimode fiber, which can form a diffraction-limited focus at any position within the fiber output field of view, making endoscopic imaging based on multimode fiber possible. It is usually necessary to calibrate the multimode fiber in advance, that is, measure its transmission matrix (TM), to obtain the optimal modulation wavefront and control the multimode fiber to perform laser scanning. Due to the short coherence length of the pulsed laser source, it is difficult to form effective off-axis interference. The coaxial holographic method is often used to measure the complex-valued TM, which is prone to producing dark spots. Alternatively, the real-valued intensity TM is obtained based on the output intensity measurements, which can only be used for binary amplitude modulation. With this method the focusing efficiency is not high, which affects the imaging quality. Therefore, a non-interferometric complex-valued TM measurement method of multimode fiber for high-resolution photoacoustic endoscopic imaging applications would be a useful invention.

### SUMMARY OF THE INVENTION

Aspects of the present invention provide a minimally-invasive multimode fiber-based photoacoustic endo-microscope enabling improved tissue imaging, including improved deep-brain imaging and the provision of neuroscience research data. This is accomplished with a nonconvex optimization method for optimum retrieval of the transmission matrix of a multimode fiber, used for non-interferometric calibration under a pulsed laser source with short coherence length. The imaging apparatus uses a single multi-mode optical fiber empowered by wavefront shaping technology as the endoscopic probe for diffraction-limited point-scanning imaging, and a highly sensitive non-focused fiber-optic ultrasonic sensor for photoacoustic signal detection. The invention thus provides lensless and high-fidelity imaging.

The invention enables the creation of an ultrathin endoscope with the imaging probe's footprint (usually on the order of 100 μm) being less than the typical cerebrovascular space (approximately 200 μm), which provides photoacoustic endomicroscopic imaging capability with high resolution and minimal invasiveness. The imaging probe of such an endoscope can be as simple as a multimode optical fiber, which reduces cost and improves efficiency, enabling in-usage replacement by standard users. This can also enable in-use calibration without access to the distal fiber side, allowing recalibration without removal of the imaging probe if required.

An aspect of the invention is a method comprising the steps of non-interferometric measurement of the transmission matrix for an MMF-PAEM apparatus as described in detail below. After such calibration, the method further comprises heating an imaging target with a laser beam, the laser beam passing through a wavefront shaping system and a fiber imaging probe; detecting a photoacoustic signal generated by the imaging target; processing the photoacoustic signals to create a photoacoustic image; and displaying the photoacoustic image.

Another aspect of the invention is an apparatus of implementing lensless multimode optical fiber-based photoacoustic endomicroscopy. It comprises a laser source producing a laser beam; a digital micromirror device (DMD) along with a 4f lens system composed of a first lens, an aperture and a second lens, which work together for high-speed, phase-only modulation of the incident light; a reflector and a first objective lens to relay and couple light into a step-index multimode optical fiber that acts as an lensless imaging probe; a non-interferometric calibration module used prior to imaging and removed after that; a fiber imaging module comprising the single multimode fiber, an imaging target which, when struck by the fiber-delivered laser pulse, is heated and produces a photoacoustic signal due to thermal expansion, and a fiber-optic ultrasonic sensor which is placed above the target and detects the photoacoustic signal.

Yet another aspect of the invention is an apparatus comprising a processor coupled to a memory, a fixed storage system, an MMF-PAEM apparatus, and a display, wherein the fixed storage is configured to store an instruction implementing the method set forth above, and the processor is configured to execute the instruction stored in the memory.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a flowchart showing the steps of the basic method of the invention.

FIG. 2 is an abstract schematic of an apparatus of lensless multimode fiber-based photoacoustic endomicroscopy of the invention.

FIG. 3 is sample imaging data produced by the method and apparatus of the invention.

FIG. 4 is an abstract schematic of an apparatus for implementing the method of the invention with a processor, memory, and other external components.

### DETAILED DESCRIPTION OF THE EMBODIMENTS

Reference will now be made in detail to several embodiments of the invention that are illustrated in accompanying drawings. Whenever possible, the same or similar reference numerals are used in the drawings and the description to refer to the same or like parts or steps. The drawings are in simplified form and are not to precise scale. For purposes of convenience and clarity only, directional terms such as top, bottom, left, right, up, down, over, above, below, beneath, rear, and front, can be used with respect to the drawings. These and similar directional terms are not to be construed to limit the scope of the invention in any manner. The words attach, connect, couple, and similar terms with their inflectional morphemes do not necessarily denote direct or intermediate connections, but can also include connections through mediate elements or devices.

The method of the invention uses laser beam(s) to illuminate targets and/or generate interference patterns. As is known to persons of ordinary skill in the art, laser beams are propagated through a medium or mediums such as a gas, a liquid, or a solid. Where a particular medium is specified for a particular point on or length of the beam path (s,) that medium is required for the best practice of the invention unless otherwise specified. Where a particular medium is not specified, any medium (gas, liquid, solid) can be used to propagate the laser beam (s.) Similarly, if a beam control means such as a lens, a prism, a polarizer, a reflector, or an optical fiber is described, unless otherwise specified such means can use any reasonable material (lenses may be glass or plastic, reflectors may be glass mirrors or bare reflective surfaces, et cetera.)

The method of the invention will first be described in general, and then particular aspects will be described in relation to the accompanying drawings.

First, a transmission matrix or “TM” recovery process for non-interferometric pre-calibration of multimode optical fiber is performed, including the following simplified steps:

In a data collection step, a series of binary computer-generated holograms encoding random phase patterns are loaded onto a digital micromirror device. As the digital micromirror device plays the calibration patterns, a camera is triggered synchronously to collect the multimode fiber generated speckle images, from which a series of known input wavefronts (collectively the “probing matrix” or “PM”) and the corresponding output amplitude measurement matrix are obtained.

In a TM recovery problem step, the two ends of the equation connecting the input phase and the output amplitude are conjugated to satisfy the form of the general phase recovery problem. In particular, for convenience of description, the problem can be divided into column-wise subproblems. Knowing the probing matrix and the amplitude measurement vector at each output mode, the corresponding TM row vector (conjugate transposed one) is recovered.

In a spectral initialization step, a subset is constructed that contains the indexes of the first several largest elements in the amplitude measurement. After conjugate transposition, the row vectors in the probing matrix corresponding to these larger output measurement values are regarded as direction vectors. Maximizing the correlation to the direction vector can be used to initialize the TM signal.

In a gradient descent step, the TM signal gradient of the least squares error between the calculated and the measured speckle amplitude values is solved, after which the gradient descent is used to iteratively approach the optimal TM solution. Specifically, this gradient descent process consists of the following two sub-steps:

In an “artificial heating” gradient descent sub-step, for the first ⅔ of total iterations, use the “artificial heating” gradient, that is, the gradient of the least square error between the calculated speckle amplitudes and the measured amplitudes' square, to perform gradient descent; and

In a normal gradient descent sub-step, for the last ⅓ of total iterations, use the normal gradient for gradient descent.

After completing the gradient descent, the method outputs the optimized TM signal. Note that the above-mentioned TM recovery process is not limited to recovering only one row of TM. Due to the natural parallelism of matrix multiplication, the above-mentioned spectral initialization and gradient descent are suitable for parallel recovery of multiple rows of TM. This method does not require an additional reference optical path for the calibration of multimode fiber, simplifying the calibration apparatus. It allows to measure the complex-valued TM of the multimode fiber, which is used to obtain the optimal wavefront and control focus scanning through the multimode fiber.

The invention features a nonconvex TM retrieval algorithm, which allows recovering the TM of a multimode fiber from the output intensity measurements, avoiding the need of a reference arm. The non-interferometric TM calibration process is described below.

Let the PM for controlling the incident wavefront be X∈ [symbol] ^{N×P}, representing the input N×1 vector for P measurements; the corresponding output speckle amplitude matrix is Y∈ [symbol] ^{M×P}, representing M×1 measurement vectors for P times; this multimode fiber input-output relationship can be expressed by the TM (A∈ [symbol] ^{M×N}): Y=|A·X|.

Since the camera loses phase information when measuring the output light field, the transmission matrix A needs to be solved through a phase retrieval algorithm. The conjugate transpose of both ends of the above equation is: Y^{H}=|X^{H} A^{H}|, considering

$${Y}^{H} = [ {y}_{1} , … {y}_{i} , … , {y}_{M} ] , {y}_{i} ∈ {ℝ}_{+}^{P} , {A}^{H} = [ {a}_{1} , … , {a}_{i} , … , {a}_{M} ] ,$$

where aᵢ∈ [symbol] ^{N}. The TM recovery problem can be divided column-by-column into sub-problems of recovering yᵢ=|X^{H}aᵢ|, i=1, . . . , M for M rows of TM.

The detailed steps of the proposed nonconvex optimization TM recovery algorithm are as follows:

Step 1: Determine that the input data includes P amplitude measurements corresponding to a certain output mode y={yⱼ}_{1≤j≤P} and the probing matrix X∈ [symbol] ^{N×P}. The input parameters include the total number of iterations T, the step size μ=3, the weighting parameters β=5, the potential of the subset |S|=└3P/13┘, and the index α=0.5.

Step 2: Spectral initialization of the TM signal. First construct a subset S containing the indexes of the first |S| largest elements in the amplitude measurement, and the row vectors in X^{H} corresponding to the first |S| larger output measurement values are regarded as the direction vectors. By maximizing the correlation to these direction vectors, the TM signal can be initialized. This can be obtained by solving for the unit principal eigenvectors ã⁰ of the weighted Hermitian matrix,

$$H = X \cdot diag ( {\tilde{y}}_{1}^{α} , {\tilde{y}}_{2}^{α} , … {\tilde{y}}_{p}^{α} ) \cdot {X}^{H} ,$$

where

$${\tilde{y}}_{j}^{α} = \{ \begin{matrix}{y}_{j}^{α} , & j ∈ S \\ 0 , & otherwise\end{matrix} .$$

In particular, the unit principal eigenvector can be solved quickly through the power method, and the result ã⁰ can be further scaled to obtain the initialized TM signal

$${a}^{0} = \sqrt{{\sum }_{j = 1}^{P} {y}_{j}^{2} / P} \cdot {\tilde{a}}^{0} .$$

Step 3: Perform gradient descent to further optimize the initialized TM signal. Specifically, the gradient descent process can be divided into the following two sub-steps:

Substep 3a: For the first ⅔ iterations

$$0 \leq t \leq ⌊ \frac{2}{3} T ⌋ - 1 ,$$

use the “artificial heating” gradient, that is, the gradient of the least square error between the calculated speckle amplitude value (X^{H} multiplied by the TM signal a^{t}) and the measured speckle amplitudes' square for gradient descent,

$${a}^{t + 1} = {a}^{t} - μ \cdot \frac{1}{P} X [ w ∘ ( {X}^{H} {a}_{t} - {y}^{2} ∘ \frac{{X}^{H} {a}^{t}}{\vert {X}^{H} {a}^{t} \vert} ) ] ;$$

Substep 3b: For the last ⅓ of the iterations

$$⌊ \frac{2}{3} T ⌋ \leq t \leq T - 1 ,$$

use the normal gradient, that is, the gradient of the least square error between the calculated speckle amplitude value (X^{H} multiplied by the TM signal a^{t}) and the measured speckle amplitude value for gradient descent,

$${a}^{t + 1} = {a}^{t} - μ \cdot \frac{1}{P} X [ w ∘ ( {X}^{H} {a}^{t} - y ∘ \frac{{X}^{H} {a}^{t}}{\vert {X}^{H} {a}^{t} \vert} ) ] ;$$

where the weight w=|X^{H} a^{t}| [symbol] (|X^{H}a^{t}|+βy), and the symbol [symbol] represents element-wise division.

Step 4: Output the optimized TM signal. In particular, the above-mentioned TM retrieval process is not limited to retrieving a single row of TM at a time, which supports parallel recovery of multiple TM rows.

By referring to the provided drawings and the above disclosure, the various aspects of the invention can be easily understood.

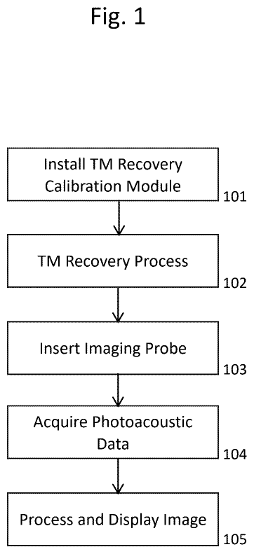

FIG. 1 shows the steps of the method of the invention.

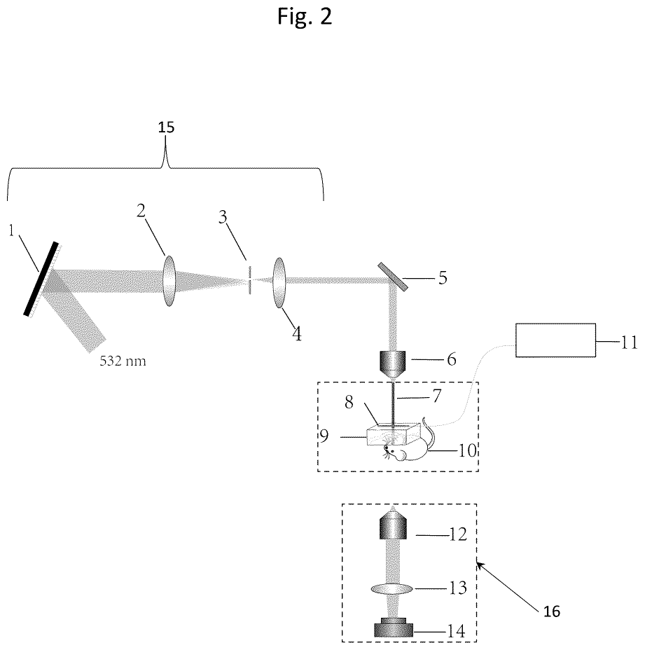

In Step 101, the TM recovery calibration module (see FIG. 2) is installed if it is not already installed.

In Step 102, the TM recovery process as described above is performed, and the calibrated TM is stored for later steps.

In Step 103, the TM recovery calibration module is removed, and the multimode fiber imaging probe is inserted into the test subject.

In Step 104, photoacoustic data is acquired by controlling focus-scanning through the fiber probe and recording the resulting photoacoustic signals with the fiber-optic ultrasonic sensor (not shown, see FIG. 2.)

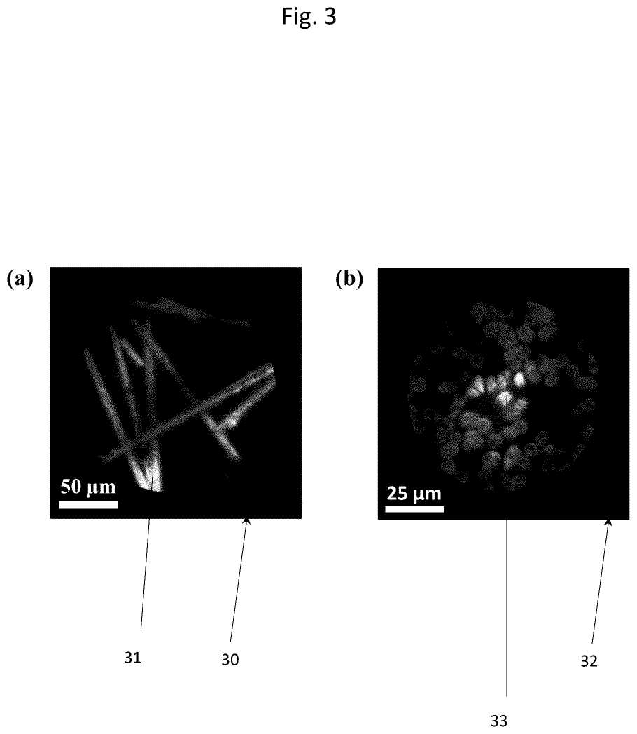

In Step 105, the photoacoustic data collected by the fiber sensor is processed and to form a photoacoustic image for display (see FIG. 3). Through an appropriate host computer program control, the acquired analog signals by the fiber-optic ultrasonic sensor during the focus-scanning process can be sampled and processed by a high-speed data acquisition and frequency demodulation module to produce effective photoacoustic data and form the tissue images via maximum amplitude projection algorithm.

FIG. 2 shows an experimental configuration of lensless multimode fiber-based photoacoustic endomicroscopy of the invention. The wavefront shaping module 15 comprising a digital micromirror device 1 and a 4F lens system (including first lens 2, aperture 3, and second lens 4), performs high-speed phase-only modulation on the incident light from an appropriate pulsed laser source, for example a pulsed 532 nm laser source (not shown) The binary computer-generated holograms (CGHs) based on the Lee hologram technique are pre-loaded onto the onboard memory of the digital micromirror device 1, for high-speed refreshing of the modulation patterns. The 532 nm pulse laser beam is expanded before being incident on the digital micromirror device 1.

After being modulated, the working beam passes through the first lens 2 and generates three orders of frequency spectrum such as +1^{st}, 0^{th} and −1^{st} on the back focal plane. The −1^{st} order can be selected through the aperture 3 on the Fourier plane, and then collimated by the second lens 4. This wavefront shaping module encodes the required phase after the second lens 4. It is preferred that digital micromirror device 1 have a kHz-scale refresh rate to achieve high-speed and high-efficiency wavefront modulation. Wavefront shaping module 15 is used to quickly produce input-output data for the pre-calibration of multimode fiber, and to control high-speed laser scanning through the multimode fiber for imaging at video rate.

The modulated and collimated light is reflected by reflector 5 into first objective lens 6, which couples the light into fiber imaging probe 7. Fiber imaging probe 7 is a step-index multimode fiber with a typical outer diameter of ˜100 microns. The fiber imaging probe is used to transmit and scan the focused beam in ways which will be familiar to persons having ordinary skill in the art.

The photoacoustic signal acquisition and processing system includes a fiber-optic ultrasonic sensor 8 based on a dual-polarized fiber laser (not shown) and a frequency demodulation and data acquisition module 11. By playing the modulation patterns that encode the phase conjugation of different rows of the measured TM at high speed, the multimode fiber can be controlled to perform high-speed focus scanning. As the wavefront shaping module controls the fiber to scan the focused beam, the ultrasonic waves produced by laser excitation and heating of the imaging target 10 are received in a non-focused manner by the fiber-optic ultrasonic sensor 8 above the imaging target 10 through sink 9. The frequency demodulation and data acquisition module 11 demodulates the change in the frequency of the beat signal and sends the corresponding data to the control circuit (not shown). This enables detection of photoacoustic signal amplitude and collection of photoacoustic data. The use of fiber-optic ultrasound sensor 8, which is placed above the imaging target, for receiving photoacoustic signal in a side-viewing and non-focused manner enables a large detection range, a high detection sensitivity, and a reduced probe size.

Second objective lens 12, third lens 13, and camera 14 form the TM recovery calibration module, which is used in Steps 101 and 102 of FIG. 1.

In an alternate embodiment of the invention (not shown,) an incremental calibration targeting device, such as a variably polarized optical filter/reflector, can be included in the imaging probe to provide a variably available fixed reference. This variably available fixed reference can provide an online calibration method for the multimode fiber of the imaging probe without the access to distal fiber end, so that recalibration of the imaging probe can be performed if it is perturbed during use.

FIG. 3 shows photoacoustic endoscopic imaging of carbon fiber filaments 31 in first sample image 30 and mouse red blood cell 33 in second sample image 32, both with an imaging resolution of approximately 2 μm. The imaging results show good contrast and signal-to-noise ratio, which demonstrate the effectiveness of the method and the implementing apparatus of the present invention.

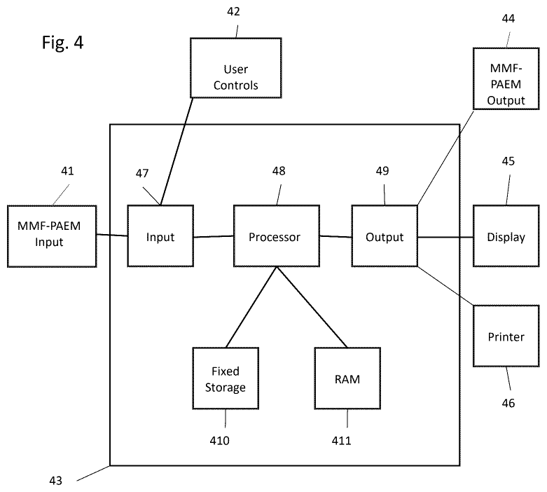

FIG. 4 shows a block diagram of apparatus for implementing the imaging method of FIG. 1. User controls 42 and MMF-PAEM input 41 are connected to an input system 47, which could comprise a Bluetooth® connection, a USB connection, a proprietary hardwired or wireless system, or any other means of connecting them as desired. MMF-PAEM input 41 can comprise one or more input sources from an MMF-PAEM apparatus, such as a camera, a digital micromirror device, a fiber imaging probe, a fiber-optic ultrasonic sensor, an optical or mechanical measurement system, et cetera. Input system 47 (which can also constitute two or more separate systems, one for each input source) sends the user control inputs and the MMF-PAEM apparatus input to processor 48. Fixed storage 410 (which could be a hard drive, a solid-state drive, flash RAM, or any other desired means of persistently storing information) and/or random-access memory (RAM) 411 contain(s) a software program or “instruction” having multiple executable code elements embodying the method of the invention which are executed by processor 48. Note that processor 48 could compromise a CPU, a GPU, a proprietary processor, or any reasonable combination thereof. Input data and processing data generated while applying the various steps of the method of the invention are also stored in RAM 411 and/or fixed storage 410. Once the inputs have been processed by processor 48 and the final generated task outputs stored in fixed storage 410 and/or RAM 411 (or offloaded to cloud storage, portable storage, or otherwise stored in final form for review) the final generated task outputs can be displayed on display 45, printed on printer 46, and/or sent to MMF-PAEM apparatus output 64, which can control one or more mechanisms related to the MMF-PAEM apparatus.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. An apparatus for high-fidelity lensless multimode fiber-based photoacoustic endomicroscopy, comprising:
a laser source producing a laser beam;
a digital micromirror device which can reflect and modulate the laser beam;
a first lens which can converge the laser beam and produce Fourier transform of the modulated beam;
an aperture which can filter the −1^{st} order of the frequency spectrum of the modulated beam, which then passes through a second lens for inverse Fourier transform and beam collimation;
a reflector which can reflect the laser beam to a first objective lens;
a fiber imaging probe communicating with the first objective lens, the fiber imaging probe comprising a multimode fiber;
an imaging target which, when struck by the laser beam, is heated and produces a photoacoustic signal caused by thermal expansion; and
a fiber-optic ultrasonic sensor which detects the photoacoustic signal.

2. The apparatus for high-fidelity lensless multimode fiber-based photoacoustic endomicroscopy of claim 1, further comprising:
a digital modulator which can modulate the laser beam into a working beam and a residual beam.

3. The apparatus of claim 2 wherein the digital micromirror device is also the digital modulator.

4. The apparatus of claim 1, further comprising:
a variable reflector communicating with the fiber-optic probe such that the laser beam can be reflected back to the fiber imaging probe.

5. The apparatus of claim 2, further comprising:
a variable reflector communicating with the fiber-optic probe such that the working beam can be reflected back to the fiber imaging probe.

6. The apparatus of claim 3, further comprising:
a variable reflector communicating with the fiber-optic probe such that the working beam can be reflected back to the fiber imaging probe.

7. The apparatus of claim 2, further comprising:
a calibration target having a group of fixed calibration patterns;
a control circuit which stores the fixed calibration patterns;
a calibration sensor which can detect the real-time intensity patterns generated by illuminating the calibration target with the laser beam; and
a calibration circuit which can perform the calibration of the target using a predetermined phase retrieval principle for empowering the apparatus.

8. A method for high-fidelity lensless multimode fiber-based photoacoustic endomicroscopy, comprising the steps of:
retrieving a complex-valued transmission matrix for an MMF-PAEM apparatus;
modulating a laser beam with a wavefront shaping module;
performing focus scanning through a multimode fiber imaging probe for the excitation of a plurality of photoacoustic signals on an imaging target;
detecting the photoacoustic signals generated by the imaging target with a side-viewing fiber-optic ultrasound sensor;
processing the photoacoustic signals to create a photoacoustic endomicroscopic image; and
displaying the photoacoustic endomicroscopic image.

9. The method for high-fidelity lensless multimode fiber-based photoacoustic endomicroscopy of claim 8, wherein the step of retrieving the transmission matrix further comprises the steps of:
storing a group of fixed calibration patterns in a control circuit;
illuminating the calibration target with the laser beam to produce a plurality of intensity measurements; and
performing the calibration of the imaging target using a predetermined phase retrieval principle.

10. An apparatus, comprising a processor coupled to a memory, a fixed storage system, an MMF-PAEM apparatus, and a display, wherein the fixed storage is configured to store an instruction, and the processor is configured to execute the instruction stored in the memory for:
retrieving a transmission matrix for an MMF-PAEM apparatus;
modulating a laser beam with a wavefront shaping module;
performing focus scanning through a multimode fiber imaging probe for the excitation of a plurality of photoacoustic signals on an imaging target;
detecting the photoacoustic signals generated by the imaging target via a fiber-optic ultrasound sensor;
processing the photoacoustic signal to create a photoacoustic endomicroscopic image; and
displaying the photoacoustic endomicroscopic image on the display.
